(19) United States
(12) Patent Application Publication
(10) Pub. No.: US 2025/0050080 A1
(43) Pub. Date: Feb. 13, 2025

(54) DEVICES, ASSEMBLIES, AND METHODS FOR DELIVERING A FLUID PREPARATION INTO A GASTROINTESTINAL TRACT

(71) Applicant: Rani Therapeutics, LLC, San Jose, CA (US)

(72) Inventor: Mir IMRAN, Los Altos Hills, CA (US)

(73) Assignee: Rani Therapeutics, LLC, San Jose, CA (US)

(21) Appl. No.: 18/810,258

(22) Filed: Aug. 20, 2024

Related U.S. Application Data
(63) Continuation of application No. PCT/US2023/013278, filed on Feb. 17, 2023.
(60) Provisional application No. 63/312,354, filed on Feb. 21, 2022.

(51) Int. Cl. A61M 31/00; A61M 5/20

(52) U.S. Cl. CPC A61M 31/00; A61M 5/2046; A61M 2210/1017

(57) ABSTRACT

A delivery assembly for an ingestible device to deliver a fluid preparation into a GI lumen wall or surrounding tissue of a subject includes a housing, a piston, a needle, and a membrane. The housing defines a chamber. The piston is movably disposed in the chamber. The needle is coupled to the piston. The membrane is coupled to the piston such that the membrane and the piston cooperatively define a reservoir for containing the fluid preparation.

### CROSS-REFERENCE TO RELATED APPLICATIONS

This application claims the benefit of and priority to U.S. Provisional Application No. 63/312,354, filed on Feb. 21, 2022, the entire contents of which are hereby incorporated by reference herein.

### BACKGROUND

A therapeutic agent such as a drug may be administered to a subject by ingestion or through parenteral injection (e.g., subcutaneously, intramuscularly, or intravenously) to provide a desired therapeutic effect. However, these routes of administration have some disadvantages. For example, some therapeutic agents such as large (macro) molecules are not suitable for delivery by ingestion because of enzymatic breakdown of these molecules in the gastrointestinal (GI) tract of a subject. Other types of therapeutic agents may otherwise be poorly tolerated within the GI tract resulting in low systemic uptake. With parenteral injections, subjects may experience pain and inconvenience with administration which can significantly impact compliance and quality of life.

To address these disadvantages, ingestible devices have been proposed to orally deliver a solid form of a therapeutic agent into a lumen wall or surrounding tissue of the GI tract. However, solid dosage forms may be limited to relatively small dosages (e.g., a few milligrams). Thus, using solid dosage forms may be disadvantageous for therapy regimens that require higher dosages due to, for example, frequency of dosing, convenience, or cost.

Accordingly, there is a need for devices which can deliver a broad range of dosages of a therapeutic agent and can address one or more of the disadvantages associated with conventional oral and parenteral routes of administration.

### SUMMARY

Embodiments of the present disclosure relate generally to devices, assemblies, and methods for delivering a fluid preparation into the GI tract (e.g., a GI lumen wall or surrounding tissue) of a subject. The devices and assemblies are structured to deliver up to about 250 microliters (μl) or more of a fluid preparation which may include a dosage of up to about 75 milligrams (mg) or more of at least one therapeutic agent. In this manner, the disclosed devices, assemblies, and methods can allow for efficient delivery of a broad range of dosages of therapeutic agents for most therapeutic regimens.

In one aspect, a delivery assembly for an ingestible device to deliver a fluid preparation into a GI lumen wall or surrounding tissue of a subject includes a housing, a piston, a needle, and a membrane. The housing defines a chamber. The piston is movably disposed in the chamber. The needle is coupled to the piston. The membrane is coupled to the piston such that the membrane and the piston cooperatively define a reservoir for containing the fluid preparation.

In another aspect, an ingestible device for delivering a fluid preparation into a GI lumen wall or surrounding tissue of a subject includes an expandable member and a delivery assembly coupled to, and disposed in, the expandable member. The delivery assembly includes a housing, a piston, a needle, a membrane, and a fluid preparation. The housing defines a chamber. The piston is movably disposed in the chamber. The needle is coupled to the piston. The membrane is coupled to the piston such that the membrane and the piston cooperatively define a reservoir. The fluid preparation is disposed in the reservoir and includes at least one therapeutic agent.

In another aspect, a device includes a tissue piercing member having an elongated section and a tapered section extending from the elongated section to a distal end. The elongated section defines a longitudinal opening extending therethrough. The tapered section defines a slotted opening extending through a side wall of the tapered section to the longitudinal opening. The slotted opening is for discharging a fluid preparation from the tissue piercing member.

In another aspect, a method for delivering a fluid preparation into a GI lumen wall or surrounding tissue of a subject includes ingesting, by the subject, an ingestible device as described herein. The ingestible device includes an expandable member and a delivery assembly coupled to, and disposed in, the expandable member. The delivery assembly includes a housing, a piston, a needle, a membrane, and a fluid preparation. The housing defines a chamber. The piston is movably disposed in the chamber. The needle is coupled to the piston. The membrane is coupled to the piston such that the membrane and the piston cooperatively define a reservoir. The fluid preparation is disposed in the reservoir and includes at least one therapeutic agent.

In another aspect, use of an ingestible device for delivering a fluid preparation into a GI lumen wall or surrounding tissue of a subject includes ingesting, by the subject, an ingestible device as described herein. The ingestible device includes an expandable member and a delivery assembly coupled to, and disposed in, the expandable member. The delivery assembly includes a housing, a piston, a needle, a membrane, and a fluid preparation. The housing defines a chamber. The piston is movably disposed in the chamber. The needle is coupled to the piston. The membrane is coupled to the piston such that the membrane and the piston cooperatively define a reservoir. The fluid preparation is disposed in the reservoir and includes at least one therapeutic agent.

In another aspect, there are provided methods of preparing an ingestible device for delivering a therapeutic agent into a GI lumen wall or surrounding tissue of a subject, comprising filling a fluid preparation comprising a therapeutic agent into a delivery assembly as described herein.

In one or more embodiments of any of the foregoing aspects, the expandable member includes a balloon.

In one or more embodiments of any of the foregoing aspects, the device further includes an ingestible enclosure, wherein the expandable member and the delivery assembly are disposed in the ingestible enclosure.

In one or more embodiments of any of the foregoing aspects, the device further includes a plurality of reactants disposed in the expandable member for generating a gas pressure to cause the expandable member to expand within a GI lumen. In one or more embodiments, the generated gas pressure is applied against the membrane to cause the piston to move axially relative to the housing.

In one or more embodiments of any of the foregoing aspects, the piston further defines a needle cavity, and the needle is slidably coupled to the piston in the needle cavity.

In one or more embodiments of any of the foregoing aspects, the delivery assembly further includes a seal coupled to the piston between the needle cavity and the reservoir to define a substantially fluid-tight seal therebetween.

In one or more embodiments of any of the foregoing aspects, the needle includes a first end structured to penetrate the GI lumen wall and a second end structured to pierce the seal in response to a resistance applied to the needle from the GI lumen wall.

In one or more embodiments of any of the foregoing aspects, the membrane comprises a flexible material such that the generated gas pressure causes the membrane to deform to expel the fluid preparation through the needle upon the needle piercing the seal.

In one or more embodiments of any of the foregoing aspects, the device is structured such that the first end of the needle penetrates the GI lumen wall before the second end of the needle pierces the seal.

In one or more embodiments of any of the foregoing aspects, the device is structured such that the first end of the needle penetrates the GI lumen wall substantially simultaneously with the second end piercing the seal.

In one or more embodiments of any of the foregoing aspects, the needle is structured to penetrate through the GI lumen wall into a peritoneum or peritoneal cavity of the subject for discharging the fluid preparation therein.

In one or more embodiments of any of the foregoing aspects, the membrane has a surface profile that is complementary to an inner surface profile of the piston which defines the reservoir.

In one or more embodiments of any of the foregoing aspects, the reservoir defines a volume for containing up to about 250 μl of fluid. In one or more embodiments, the reservoir defines a volume for containing about 50 μl to about 200 μl of fluid.

In one or more embodiments of any of the foregoing aspects, the piston includes a fill port for filling the reservoir with the fluid preparation.

In one or more embodiments of any of the foregoing aspects, the delivery assembly further includes a fluid preparation disposed in the reservoir, wherein the fluid preparation includes at least one therapeutic agent. In one or more embodiments of any of the foregoing aspects, the at least one therapeutic agent is one or more selected from an immunosuppressive drug, a chemotherapy drug, a central nervous system (CNS) drug, an antidiabetic drug, an enzyme replacement therapy (ERT) drug, an antibody, a hormone, insulin, an incretin or a combination thereof, or an oligonucleotide.

In one or more embodiments of any of the foregoing aspects, the tissue piercing member further includes a tip coupled to, or integrally formed with, the tapered section at the distal end. In one or more embodiments, the tip includes a material that is harder than a material of the tissue piercing member.

In one or more embodiments, the device is ingestible.

The foregoing general description and following detailed description are provided by way of example and are intended to provide further explanation of the disclosure as claimed, without limiting the disclosure or the claims. Other objects, advantages, and novel features will be readily apparent to those skilled in the art from the following brief description of the drawings and detailed description of the disclosure.

### BRIEF DESCRIPTION OF THE DRAWINGS

FIG. 1 illustrates an embodiment of a device for delivering a fluid preparation into a GI tract of a subject in block diagrammatic form.

FIG. 2 illustrates a cross-sectional view of an embodiment of the device of FIG. 1.

FIG. 3 illustrates a detail view of a portion of the device of FIG. 2 including a delivery assembly.

FIG. 4 illustrates the device of FIG. 3 in a second state when the device has reached a desired location in the GI tract to deliver the fluid preparation.

FIG. 5 illustrates a cross-sectional view taken along line 5-5 in FIG. 3.

FIG. 6 illustrates a method of delivering a fluid preparation into the GI tract of a subject using the devices and assemblies of the present disclosure.

FIGS. 7-8 illustrate a tissue piercing member suitable for use in a device as described herein, such as the ingestible device of FIG. 1.

### DETAILED DESCRIPTION

Before discussing details of the devices, assemblies, and methods of the present disclosure, a few conventions are provided for the convenience of the reader.

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural references unless the context clearly dictates otherwise. Reference to an object in the singular is not intended to mean “one and only one” unless explicitly so stated, but rather “one or more.”

As used herein, a phrase in the form “A/B” or in the form “A and/or B” means (A), (B), or (A and B); a phrase in the form “at least one of A, B, or C” means (A), (B), (C), (A and B), (A and C), (B and C), or (A, B, and C).

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

The term “component” refers herein to one item of a set of one or more items that together make up a device, a composition, or a system under discussion. A component may be in a solid, powder, gel, plasma, fluid, gas, or other constitution. For example, a device may include multiple solid components which are assembled together to structure the device and may further include a fluid component that is disposed in the device. For another example, a composition may include a single component, or two or more components which are mixed together to make the composition. A composition may be in the form of a fluid, a slurry, a powder, or a solid (e.g., in a condensed or a consolidated form such as a tablet or microtablet). A device or system can include one or more compositions and/or one or more other components.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, Ph, species, diet, health, gender, age, ancestry, disease, or tissue damage); it is to be understood that actual tolerances and environmental conditions before and/or after delivery can affect characteristics so that different components, devices, compositions, or systems with a same design can have different actual values with respect to those characteristics. Design encompasses also variations or modifications before or after manufacture.

The term “structured” or a grammatical variation thereof (e.g., “structure” or “structuring”) refers herein to a component, device, composition, or system that is manufactured according to a concept or design or variations thereof or modifications thereto (whether such variations or modifications occur before, during, or after manufacture) whether or not such concept or design is captured in a writing.

The term “body” refers herein to an animalia body, unless the context clearly dictates otherwise.

The term “subject” refers herein to a body into which an embodiment of the present disclosure is, or is intended to be, delivered. For example, with respect to humans, a subject may be a patient under treatment of a health care professional. The terms “individual,” “subject,” and “patient” may be used interchangeably herein, and refer to any individual animalia subject (e.g., bovine, canine, feline, equine, or human). In specific embodiments, the subject, individual, or patient is a human.

The term “fluid” refers herein to a liquid or gas, and encompasses moisture and humidity. The term “fluidic environment” refers herein to an environment in which one or more fluids are present.

The term “ingest” or a grammatical variation thereof (e.g., “ingesting”, “ingestion,” or “ingested”) refers herein to taking into the stomach, whether by swallowing or by other means of depositing into the stomach (e.g., by depositing into the stomach by endoscope or depositing into the stomach via a port).

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The term “degradation rate” or a grammatical variation thereof (e.g., “rate of degradation”) refers herein to a rate at which a material degrades. A designed degradation rate of a material in a particular implementation can be defined by a rate at which the material is expected to degrade under expected conditions (e.g., in physiological conditions) at a target delivery site. A designed degradation time for a particular implementation can refer to a designed time to complete degradation or a designed time to a partial degradation sufficient to accomplish a design purpose (e.g., breach). Accordingly, for example, a designed degradation time can be specific to a component and/or specific to expected conditions at a target delivery site. A designed degradation time can be short or long and can be defined in terms of approximate times, maximum times, or minimum times.

The term “substantially” is used herein to describe and account for small variations which may result from, for example, a manufacturing or assembly process. For example, when used in conjunction with a numerical value, the terms can refer to a variation in the value of less than or equal to ±10%.

The term “lumen” refers herein to the inside space of a tubular structure. Examples of lumens in a body include arteries, veins, and tubular cavities within organs.

The term “lumen wall” refers to a wall of a lumen, where the wall includes all layers from an inner perimeter to an outer perimeter of the lumen, such as, with respect to lumens in a body, the mucosa, submucosa, muscularis, serosa, and an outer wall of the lumen, with the constituent blood vessels and tissues.

The term “gastrointestinal tract” or “GI tract” refers herein to the intake/expulsion system of a body including, for example, the mouth, pharynx, esophagus, stomach, pylorus, small intestine, cecum, large intestine, colon, rectum, anus, and valves or sphincters therebetween.

The term “GI lumen” refers generally to any lumen of the GI tract (e.g., a lumen of the esophagus, stomach, small intestine, large intestine, or colon) and the term “GI lumen wall” refers to a lumen wall of a GI lumen.

As used herein, the terms “comprising”, “comprise”, “comprises”, “includes”, and “including” are intended to mean that the compositions and methods include the recited elements, but do not exclude others.

Referring generally to the Figures, disclosed herein are embodiments relating to devices, assemblies, and methods for delivering a fluid preparation into a lumen wall or surrounding tissue (e.g., a peritoneum or peritoneal cavity) of a GI tract of a subject. The devices are advantageously structured to substantially preserve one or more therapeutic agents in the fluid preparation within the GI tract for delivery into a GI lumen wall or surrounding tissue thereof. Delivery into the GI lumen wall or surrounding tissue can allow for systemic uptake of one or more therapeutic agents in the fluid preparation. Further, the fluid form of the preparation can allow for delivery of a broad range of dosages (e.g., up to about 75 mg or more) of one or more therapeutic agents for most therapeutic regimens.

As discussed below, the disclosed devices and assemblies can deliver a variety of different types of therapeutic agents, such as macromolecules that are normally unsuitable for delivery by ingestion. In this manner, the disclosed devices, assemblies, and methods can address many of the disadvantages associated with conventional oral and parenteral routes of administration.

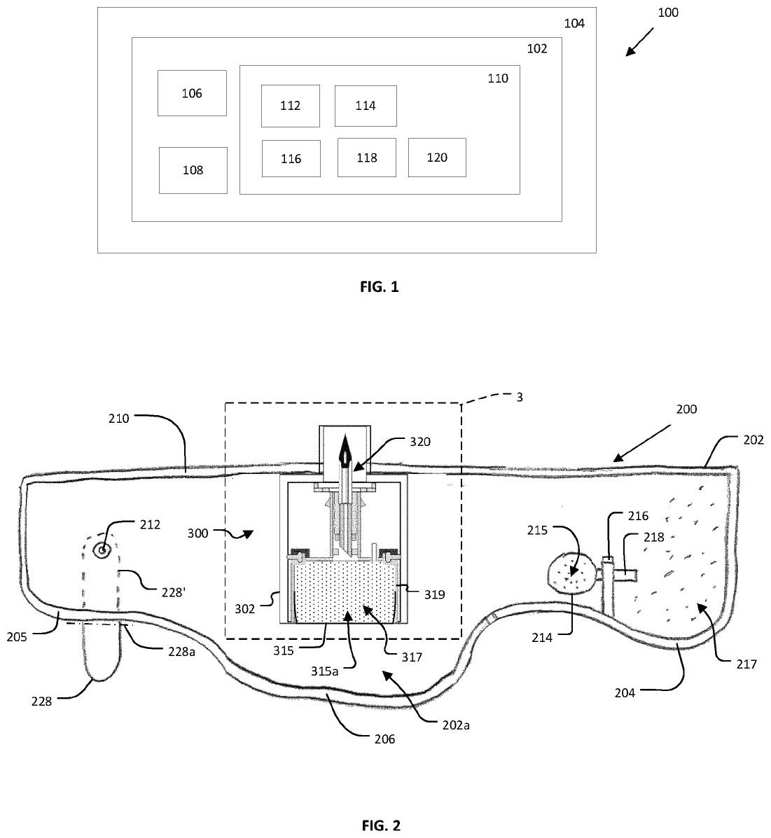

FIG. 1 illustrates in block diagrammatic form an example of a device 100 according to one or more embodiments of the present disclosure. Device 100 includes an enclosure 102, an optional outer coating 104, an expandable member 106, a release 108, and a delivery assembly 110. Delivery assembly 110 includes a housing 112, a membrane 114, a fluid preparation 116, a piston 118, and a needle 120.

According to a non-limiting example, enclosure 102 (or optionally enclosure 102 and/or outer coating 104) can degrade at a desired location in the GI tract (e.g., the stomach or small intestine) of a subject for delivery of fluid preparation 116, as described in more detail below. As described in more detail below, in response to at least partial degradation of enclosure 102 and/or outer coating 104, release 108 is activated to cause a chemical reaction within expandable member 106 resulting in the formation of a gas to cause expandable member 106 to expand. Expansion of expandable member 106 within the GI lumen causes delivery assembly 110 to be positioned proximate to (e.g., adjacent or in contact with) the lumen wall. When the internal pressure generated by the gas within expandable member 106 reaches a threshold value (e.g., upon expansion of expandable member 106), the gas pressure applied to membrane 114 causes piston 118 to move relative to housing 112 and advance needle 120 toward the GI lumen wall. The GI lumen wall applies a resistance to needle 120 to cause piston 118 to move relative to needle 120 such that needle 120 pierces a seal on piston 118 to allow fluid preparation 116 to flow to needle 120. Upon piercing the seal, the gas pressure applied against membrane 114 causes membrane 114 to expel fluid preparation 116 through needle 120 and into the GI lumen wall or surrounding tissue thereof.

Enclosure 102 and outer coating 104, if present, are structured to allow for ingestion of device 100, and to temporarily protect the contents of device 100 from degradation within one or more portions of the GI tract of a subject. Enclosure 102 may take a variety of different forms and shapes, such as a swallowable capsule (e.g., a size 00 capsule, a size 000 capsule, or other size capsule), or any other structure that is suitable for ingestion by a subject and can house or contain one or more of the components of device 100 (e.g., at least expandable member 106, release 108, and delivery assembly 110). In one or more embodiments, enclosure 102 includes two or more sections coupled (e.g., press-fit) together to define enclosure 102. For example, enclosure 102 may be structured as a capsule including a first section at least partially overlapping a second section in a press-fit arrangement to define enclosure 102. The first and second sections may be detachably coupled together so as to allow for separation of the two sections. In one or more embodiments, expandable member 106, release 108, and delivery assembly 110 are each structured to be contained within an interior of enclosure 102. Release 108 may optionally be located outside of the interior of enclosure 102, such as on an outer portion of enclosure 102.

In one or more embodiments, enclosure 102 can degrade under certain conditions. Further, different portions of enclosure 102 may be structured to degrade under different conditions or at different degradation rates depending on a target site within the GI tract for delivering fluid preparation 116. For example, a portion of, or all of, enclosure 102 may be constructed of a material that degrades in water (e.g., in the presence of water in the form of humidity or moisture in an ambient environment, such as within the body) and/or degrades when exposed to a pH level above a particular threshold or within a particular range (e.g., a pH level associated with a desired location or portion of the GI tract, such as a pH level associated with a target site within the GI tract for delivering fluid preparation 116).

Optional outer coating 104 optionally covers a portion of, or all of, enclosure 102. Outer coating 104 may include a single layer or multiple layers. The various layers may be formed of the same material or different materials. In one or more embodiments, outer coating 104 can degraded under certain conditions, as described above with reference to enclosure 102. An example of outer coating 104 is an enteric coating, such as an enteric coating that degrades in water at a given rate and/or degrades when exposed to solutions with a pH level above a particular threshold or within a particular range. Another example of outer coating 104 is a protective coating (e.g., wax), such as a coating which protects a portion of an outer surface of enclosure 102 from coming into contact with fluids or tissues (e.g., bodily tissue or fluids).

In one or more embodiments, degradation of enclosure 102 and/or outer coating 104 allows fluid (e.g., bodily fluid in the stomach or in the intestine) to enter into an interior of enclosure 102/outer coating 104 to activate release 108. Alternatively, release 108 may be located on an outer portion of enclosure 102 and degradation of outer coating 104 may expose release 108 on a surface of enclosure 102 so as to facilitate activation of release 108. Alternatively, release 108 may be located on a portion of enclosure 102 without outer coating 104, and release 108 may be structured to degrade at a different rate and/or under different conditions in the GI tract than enclosure 102.

Enclosure 102 and/or outer coating 104 may define one or more degradation areas for localized degradation of enclosure 102 and/or outer coating 104 so as to, for example, allow for controlled degradation and separation of enclosure 102. For example, outer coating 104 may be selectively applied only to certain areas of enclosure 102 (e.g., on the ends of enclosure 102) to expose a selected portion of enclosure 102 (e.g., a middle portion of enclosure 102 between the ends), thereby defining an area of enclosure 102 that can degrade at a faster rate and/or degrade sooner than other areas of enclosure 102. This controlled degradation of enclosure 102 may allow for more consistent separation of enclosure 102 to thereby allow for delivery of fluid preparation 116 into the GI lumen wall.

Expandable member 106 is a flexible and adjustable structure. In one or more embodiments, expandable member 106 is structured to expand from a collapsed state (e.g., folded, rolled, flattened) to an expanded state within a desired location of a GI lumen for delivery of fluid preparation 116. Expandable member 106 may have a variety of different shapes, sizes, and configurations for being temporarily stored in enclosure 102 and for being deployed within a lumen of the GI tract of a subject. Expandable member 106 may be structured as a balloon, a bellows/accordion structure, or any other structure that can adjust from a collapsed state to an expanded state.

Expandable member 106 defines an interior volume for containing various components of device 100. For example, expandable member 106 may include (e.g., contain) one or more reactants that are temporarily separated from each other within the interior volume. The interior volume is structured to facilitate a chemical reaction caused by mixing the reactants together (e.g., in response to activation of release 108) to form a gas to cause expansion of expandable member 106. Expandable member 106 may further include a structure for deflating expandable member 106 upon delivery of fluid preparation 116 to facilitate excretion of expandable member 106 from a subject. Expandable member 106 is structured to stretch a defined amount without perforation upon expansion.

Expandable member 106 may be formed from one or more materials. Examples of suitable materials for expandable member 106 include hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof. Expandable member 106 may include one or more layers of material. Expandable member 106 may be a monolithic structure. In other embodiments, expandable member 106 may be composed of one or more sections that are coupled (e.g., sealed or sewn) together.

Release 108 is a chemical, mechanical, electrical, electro-mechanical, electro-chemical, chemo-mechanical, or electro-mechanical-chemical structure. Release 108 is structured to cause expansion of expandable member 106 upon activation of release 108. In one or more embodiments, release 108 is structured to be activated (e.g., degrade, release, move, open) in response to a condition in the GI tract. For example, release 108 may be structured to degrade in water, such that release 108 may degrade upon contact with fluid in the GI tract. For another example, release 108 may be structured to degrade at or above a particular pH level or within a range of pH levels associated with a location in the GI tract (e.g., a pH in the stomach, a pH in the intestine.) In these and other embodiments, release 108 may be made from a biodegradable material, such as an enteric material.

Additionally or alternatively, release 108 may be structured as a latch, a clip, a cover, a plug, a coating, or any other structure that moves, opens, or otherwise releases in response to a condition in the GI tract. Release 108 may be formed from a single material or a combination of materials. Release 108 may include one or more components. In embodiments in which a plurality of components are included in release 108, the components may be co-located (e.g., co-axially) or may be physically separated from each other.

Delivery assembly 110 is a chemical, mechanical, electrical, electro-mechanical, electro-chemical, chemo-mechanical, or electro-mechanical-chemical structure. In one or more embodiments, delivery assembly 110 can include housing 112, membrane 114, fluid preparation 116, piston 118, and needle 120. In other embodiments, delivery assembly 110 can include housing 112, membrane 114, fluid preparation 116, and piston 118 without needle 120. In these embodiments, delivery assembly 110 may be structured to deliver fluid preparation 116 as a fluidic jet through a nozzle or opening on housing 112, where the fluidic jet has sufficient velocity to penetrate the GI lumen wall for delivery of fluid preparation 116.

Delivery assembly 110 is structured to be coupled to, and disposed in (partially or fully), expandable member 106. In response to expansion of expandable member 106, delivery assembly 110 is further structured to deliver fluid preparation 116 into the GI lumen wall or surrounding tissue. Device 100 may include one or more delivery assemblies 110 coupled to expandable member 106 to allow for delivery of one or more fluid preparations.

Housing 112 is structured to be coupled to, and disposed in (partially or fully), expandable member 106. Housing 112 defines one or more chambers for movably receiving piston 118 and needle 120 or a piston without a needle. Housing 112 is structured to allow a gas pressure from within expandable member 106 to be applied against membrane 114 to axially move piston 118 and needle 120 relative to housing 112. Housing 112 may include one or more components. In embodiments in which a plurality of components are included in housing 112, the components may be co-located or may be physically separated from each other.

Membrane 114 is a flexible structure. Membrane 114 is coupled to piston 118. Membrane 114 and piston 118 cooperatively define a reservoir for containing fluid preparation 116. The reservoir may define a volume for containing up to about 250 μl or more of fluid, including 250 μl of fluid. The reservoir may define a volume for containing from about 50 μl to about 200 μl of fluid including from 50 μl to 250 μl of fluid, such as 50 μl, 100 μl, 150 μl, or 200 μl of fluid, or any value therebetween, or may contain 200 μl or 250 μl of fluid, or any value therebetween. Membrane 114 is further structured to expel fluid preparation 116 from the reservoir into needle 120 in response to a pressure (e.g., from a gas generated within expandable member 106) applied against an outer surface of membrane 114.

Membrane 114 may have any suitable shape, such as cylindrical, ellipsoidal, spherical, cuboidal, or other shape. Membrane 114 may be formed from, or otherwise include, a flexible or pliable material to allow for deformation (e.g., bending/flexing by pressure) of membrane 114 to expel fluid preparation 116. Examples of suitable materials for membrane 114 include a polymeric material (e.g., polyethylene terephthalate (PET)), or other flexible material having sufficiently low moisture and gas permeability properties for use with fluid preparation 116. Membrane 114 may be formed from a single material or a combination of materials. Further, membrane 114 may include one or more layers of a material. Membrane 114 may be a monolithic structure. In other embodiments, membrane 114 may be composed of multiple sections coupled (e.g., sealed or sewn) together to define membrane 114. Membrane 114 may include one or more components. In embodiments in which a plurality of components are included in membrane 114, the components may be co-located or may be physically separated from each other.

Fluid preparation 116 is a preparation including one or more components where the preparation is intended for a therapeutic, diagnostic, or other biological purpose. Fluid preparation 116 is in a fluid form, such as a liquid, a slurry, a gel, a suspension (including a colloidal suspension), a gas, a powder, or any combination thereof. Fluid preparation 116 includes one or more components including one or more therapeutic agents, such as a protein, a peptide, a polypeptide, an antibody, a drug (e.g., one or more selected from immunosuppressive drugs (e.g., adalimumab), chemotherapy drugs, central nervous system (CNS) drugs (e.g., antiparkinson agents, antiemetic agents), antidiabetic drugs (e.g., metformin), enzyme replacement therapy (ERT) drugs), hormones (e.g., parathyroid hormone (PTH), follicle stimulating hormone (FSH), and analogues thereof), insulin, an incretin or a combination thereof (e.g., GLP-1, GLP-2, GIP, glucagon, PYY, and analogues thereof)), an oligonucleotide (e.g., antisense oligonucleotides (ASO), RNA interference (RNAi), aptamer RNAs), a DNA or SiRNA transcript, a cell, a cytotoxic agent, a vaccine or other prophylactic agent, a nutraceutical agent, a vasodilator, or a vasoconstrictor, a delivery enhancing agent, a delay agent, an excipient, a diagnostic agent, or a substance for cosmetic enhancement. Fluid preparation 116 may include a therapeutically effective amount of one or more therapeutic agents as well as suitable amounts of other components (e.g., excipients) to achieve a desired therapeutic effect in a subject.

Piston 118 is movably (e.g., slidably) disposed in housing 120. Piston 118 includes one or more cavities which cooperate with membrane 114 to define a reservoir for holding fluid preparation 116. Piston 118 is further structured to interface with needle 120 to allow needle 120 to move relative to piston 118 to selectively control a flow of fluid preparation 116 from the reservoir to needle 120. Piston 118 is further structured to advance needle 120 into the GI lumen wall or surrounding tissue in response to sufficient pressure applied to piston 118 (e.g., via a gas pressure applied to membrane 114.) Piston 118 may include one or more components. In embodiments in which a plurality of components are included in piston 118, the components may be co-located or may be physically separated from each other.

Needle 120 is coupled to piston 118 and is structured to penetrate the GI lumen wall or surrounding tissue thereof. Needle 120 may have a length sufficient to penetrate to a desired depth in a GI lumen wall or surrounding tissue of a subject. For example, needle 120 may have a length sufficient to penetrate into and through the GI lumen wall into the peritoneum or peritoneal cavity of a subject for discharging fluid preparation 116 therein. In one or more embodiments, needle 120 is movable relative to piston 118. In other embodiments, needle 120 is fixed relative to piston 118. Needle 120 includes a channel for receiving fluid preparation 116 from the reservoir and an opening for discharging fluid preparation 116 into the GI lumen wall or surrounding tissue.

Needle 120 may be at least partially, or may be fully, degradable such that needle 120 can degrade within the GI lumen or surrounding tissue upon delivery of fluid preparation 116. Suitable materials for needle 120 may include, for example, polyethylene oxide (PEO), magnesium, a polymeric material, or other materials or combinations of materials. Needle 120 may include one or more components. In embodiments in which a plurality of components are included in needle 120, the components may be co-located or may be physically separated from each other. In other embodiments, needle 120 may be substantially non-degradable or may include substantially non-degradable portions. In any embodiments, and particularly in embodiments where needle 120 is substantially non-degradable, delivery assembly 110 may be equipped with a return spring or other biasing structure to permit retraction of needle 120 from the GI lumen wall within housing 112 to prevent injury to a subject.

One or more components of device 100 (e.g., enclosure 102, outer coating 104, expandable member 106, release 108, delivery assembly 110) may be formed from, or otherwise include, one or more biodegradable materials to facilitate degradation of such components to, for example, allow for passage through the remainder of the intestinal tract of a subject after delivery of fluid preparation 116. Examples of biodegradable materials that may be suitable for use with various components of device 100 include, for example, hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof.

The preceding description is an overview of ingestible device 100. The following description with reference to FIGS. 2-8 relates to examples of various features and aspects of device 100. These examples are illustrative of, and not limiting on, device 100.

Referring to FIG. 2, a cross-sectional view of a device 200 (an embodiment of device 100) is illustrated in an unfolded and/or unrolled state. Device 200 is illustrated without enclosure 102 and optional outer coating 104. However, it should be appreciated that device 200 may be folded, rolled, and/or otherwise manipulated to be disposed within enclosure 102 (optionally provided with outer coating 104) to allow for ingestion of device 200 and subsequent delivery into the GI tract.

Device 200 includes an expandable member in the form of a balloon 202 (an embodiment of expandable member 106), a deflation valve 212, a reactant reservoir 214 containing a first reactant 215, a release 216 (an embodiment of release 108), a second reactant 217, and a delivery assembly 300 (an embodiment of delivery assembly 110.)

Still referring to FIG. 2, balloon 202 includes an inflator section 204, a deflator section 205, a lower section 206, and an elongated section 210 extending between inflator section 204 and deflator section 205. Lower section 206 extends downward (in the orientation shown in FIG. 2) between inflator section 204 and deflator section 205. The various sections of balloon 202 cooperatively define an interior 202a for containing various components of device 200. As discussed below, balloon 202 is structured to inflate at a desired location within a lumen of the GI tract (e.g., stomach, intestine) in response to the formation of a gas from a chemical reaction between first reactant 215 and second reactant 217 within interior 202a. In this way, balloon 202 can facilitate delivery of a fluid preparation 317 (an embodiment of fluid preparation 116) from delivery assembly 300 into the GI lumen wall or surrounding tissue thereof.

Balloon 202 has a size and shape to occupy a space in a GI lumen upon inflation of balloon 202 to allow for delivery of fluid preparation 317 into the GI lumen wall. For example, upon inflation of balloon 202, an outer periphery of balloon 202 (e.g., an outer periphery of lower section 206 and elongated section 210) pushes against a surface of the lumen wall. The pressure exerted by balloon 202 is sufficient to temporarily hold balloon 202 relative to the lumen wall for delivery of fluid preparation 317. Depending on an inner circumference of the lumen delivery site, lower section 206 may remain partially folded, or may extend fully, when balloon 202 is inflated. For example, if the lumen is relatively large and there is no obstruction to resist expansion of balloon 202, then balloon 202 would assume a fully inflated configuration with lower section 206 fully extended (as shown in FIG. 2.) If, however, the lumen is relatively small such that the inner lumen circumference is less than a maximum dimension of the fully inflated balloon 202, then lower section 206 would remain partially folded. In this way, balloon 202 can self-adjust to the size of a GI lumen to hold balloon 202 in position for delivery of fluid preparation 317, such that the same balloon 202 can be used for a broad range of lumen sizes (e.g., different inner circumferences.)

Deflation valve 212 is structured to cause deflation of balloon 202 upon completion of delivery of fluid preparation 317 into the GI lumen wall or surrounding tissue thereof. In this way, deflation valve 212 can facilitate passage of balloon 202 through the remainder of the GI tract to exit the anus of the subject. In the embodiment shown, deflation valve 212 is structured as a degradable plug which temporarily covers an opening leading into interior 202a. The degradable plug may be structured to degrade in response to contact with fluid in the GI tract (e.g., bodily fluid) to thereby allow gas contained in interior 202a to exit through the opening. For example, deflation valve 212 may be formed from, or include, an enteric material. Balloon 202 may include an optional flap 228 which can temporarily cover deflation valve 212 until balloon 202 is inflated to thereby prevent premature activation (e.g., degradation) of deflation valve 212. For example, flap 228 may be temporarily held (e.g., adhered, tacked, or otherwise held) in a folded position 228′ about a flap folding axis 228a. The expansion of balloon 202 can cause flap 228 to unfold from the folded position 228′ to expose deflation valve 212 and allow fluid in the GI tract to reach deflation valve 212 and cause its degradation, thereby providing an opening for gas to exit through to permit deflation.

Deflation valve 212 is shown located on deflator section 205, but deflation valve 212 may be located elsewhere on expandable member 202 according to other embodiments. Further, balloon 202 may include more than one deflation valve 212. Deflation valve 212 may be structured differently than the embodiment shown in FIG. 2. For example, in other embodiments, deflation valve 212 may be structured as a degradable and/or movable cover disposed over an opening on balloon 202. In these embodiments, degradation and/or movement of the cover away from the opening can cause gas to exit from interior 202a through the opening, thereby permitting deflation.

Reactant reservoir 214 is disposed within interior 202a and is structured to hold first reactant 215 therein and to temporarily prevent first reactant 215 from contacting second reactant 217, which is separately disposed within interior 202a. First reactant 215 may be, for example, citric acid. Second reactant 217 may be, for example, a carbonate, such as potassium bicarbonate. In other embodiments, first reactant 215 and second reactant 217 may be other types of reactants (e.g., an acid and a base) which when mixed result in the formation of a gas sufficient to inflate balloon 202. Second reactant 217 is shown disposed within interior 202a at inflator section 204 near first reactant 215. In other embodiments, first reactant 215 and second reactant 217 may be contained in other areas of balloon 202 so long as they are temporarily separated from each other.

Reactant reservoir 214 defines an interior volume for containing first reactant 215. Reactant reservoir 214 may take a variety of different forms and shapes, such as a balloon or other structure. Reactant reservoir 214 is in selective fluid communication with interior 202a via reactant conduit 218 and release 216. Release 216 is coupled to reactant conduit 218 such that upon activation (e.g., degradation) of release 216, first reactant 215 can exit from reactant reservoir 214 into interior 202a via reactant conduit 218. For example, release 216 may be in the form of a degradable plug which blocks an interior portion of reactant conduit 218 to temporarily prevent first reactant 215 from entering interior 202a. When fluid in the GI tract contacts release 216 (e.g., upon degradation of enclosure 102 and/or outer coating 104), release 216 can subsequently degrade to allow reactant conduit 218 to discharge first reactant 215 into interior 202a.

In other embodiments, device 200 includes a clip, a band, or other structure for holding a portion of balloon 202 in such a manner to temporarily define separate chambers within interior 202a for separately containing first reactant 215 and second reactant 217, respectively. For example, a portion of balloon 202 may be pinched or compressed by a degradable clip or band to temporarily define the separate chambers within interior 202a. The chambers may be substantially sealed from each other to substantially prevent first reactant 215 and second reactant 217 from mixing. The clip or band may be located on an outer portion of balloon 202 such that exposure to fluid in the GI tract (e.g., upon degradation of enclosure 102 and/or outer coating 104) can cause degradation of the clip or band and subsequent release from balloon 202. Upon release of the clip or band or like structure from balloon 202, the separate chambers are no longer substantially sealed from each other thereby allowing first reactant 215 to mix with second reactant 217 within interior 202a.

Combining first reactant 215 with second reactant 217 within interior 202a causes a chemical reaction resulting in the formation of a gas (e.g., CO₂·) The gas causes balloon 202 to expand to an inflated state within a GI lumen resulting in substantial alignment of elongated section 210 with a surface of the GI lumen wall. Substantial alignment of elongated section 210 relative to the GI lumen wall can, advantageously, help to facilitate delivery of fluid preparation 317 from delivery assembly 300 into the GI lumen wall or surrounding tissue thereof.

Still referring to FIG. 2, delivery assembly 300 is coupled to balloon 202 and is at least partially disposed within interior 202a. The delivery assembly 300 is shown to include a housing 302 (an embodiment of housing 112), a membrane 315 (an embodiment of membrane 114), fluid preparation 317 (an embodiment of fluid preparation 116), a piston 319 (an embodiment of piston 118), and a needle 320 (an embodiment of needle 120). Membrane 315 and piston 319 cooperatively define a reservoir 315a for containing fluid preparation 317.

As discussed below, the gas that pressurizes balloon 202 can pass through an opening of housing 302 to apply a pressure against membrane 315 on piston 319. When the gas pressure reaches a threshold value (e.g., upon full or partial inflation of balloon 202 within the GI lumen), the pressure causes piston 319 and needle 320 to move relative to housing 302 toward the GI lumen wall. In response to a resistance applied to needle 320 from the GI lumen wall, piston 318 moves axially relative to needle 320 to cause needle 320 to pierce a seal on piston 319 to thereby allow fluid preparation 317 to flow from reservoir 315a to needle 320. Upon piercing the seal, the gas pressure causes needle 320 to advance further into the GI lumen wall or surrounding tissue. The gas pressure also causes membrane 315 to expel fluid preparation 317 from reservoir 315a through needle 320 and into the GI lumen wall or surrounding tissue.

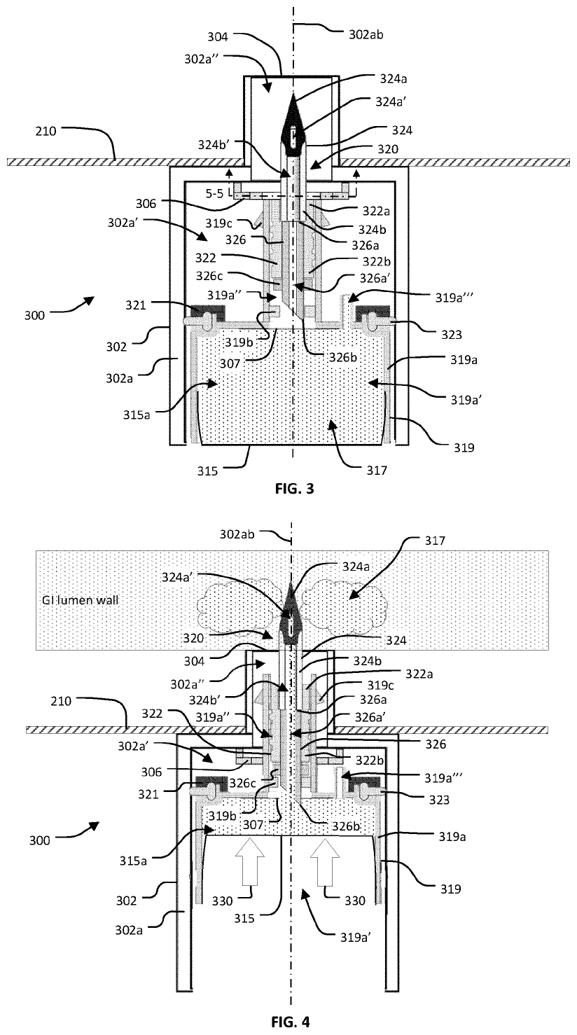

Referring to FIG. 3, a detail view of a portion of device 200 including delivery assembly 300 is illustrated. As shown in FIG. 3, delivery assembly 300 includes housing 302, a needle seal 304, a release mechanism 306, a reservoir seal 307, membrane 315, fluid preparation 317, piston 319, needle 320, a piston seal holder 321, and a piston seal 323. In the embodiment shown, needle 320 is collectively defined by a needle sleeve 322, a tissue piercing member 324, and a seal piercing member 326. In other embodiments, needle 320 may be a monolithic structure including needle sleeve 322, tissue piercing member 324, and seal piercing member 326.

Housing 302 includes a housing perimeter wall 302a which defines a piston chamber 302a′ and a needle chamber 302a″. Piston 319 is movably (e.g., slidably) disposed in piston chamber 302a′. A portion of needle 320 (e.g., tissue piercing member 324) is disposed in needle chamber 302a″. In the orientation shown in FIG. 3, needle chamber 302a″ extends from an upper end of piston chamber 302a′. Housing 302 is structured to be coupled (e.g., heat sealed, adhered) to balloon 202 (e.g., to elongated section 210 shown in FIG. 3) along housing perimeter wall 302a at the transition between piston chamber 302a′ and needle chamber 302a″. In an assembled state, a portion of housing perimeter wall 302a located above piston chamber 302a′ extends outwardly from elongated section 210 of balloon 202 for positioning needle chamber 302a″ proximate to the GI lumen wall. Piston chamber 302a′ and needle chamber 302a″ each have a substantially cylindrical shape and cooperatively define a longitudinal axis 302ab for piston 319 and needle 320 to move axially along. It should be appreciated, however, that piston chamber 302a′ and needle chamber 302a″ may have different shapes besides cylindrical (e.g., ovular, spherical) according to other embodiments. Housing 302 may further define one or more vent ports to function as a pressure relief for piston chamber 302a′ or balloon interior 202a during or after axial movement of piston 319 relative to housing 302. The vent ports may be structured to permit selective fluid communication between piston chamber 302a′ and balloon interior 202a with the GI lumen environment outside of device 200.

Needle seal 304 is coupled (e.g., adhered) to housing perimeter wall 302a at an upper end of needle chamber 302a″ to substantially prevent fluid in the GI tract from entering into needle chamber 302a″ until needle 320 (e.g., tissue piercing member 324) pierces needle seal 304. Needle seal 304 may be formed from a penetrable material (e.g., aluminum foil) to allow needle 320 to pierce through needle seal 304. In this way, needle seal 304 and housing perimeter wall 302a can cooperatively define a substantially sterile environment within needle chamber 302a″ for containing needle 320 before delivery of fluid preparation 317 into the GI lumen wall or surrounding tissue.

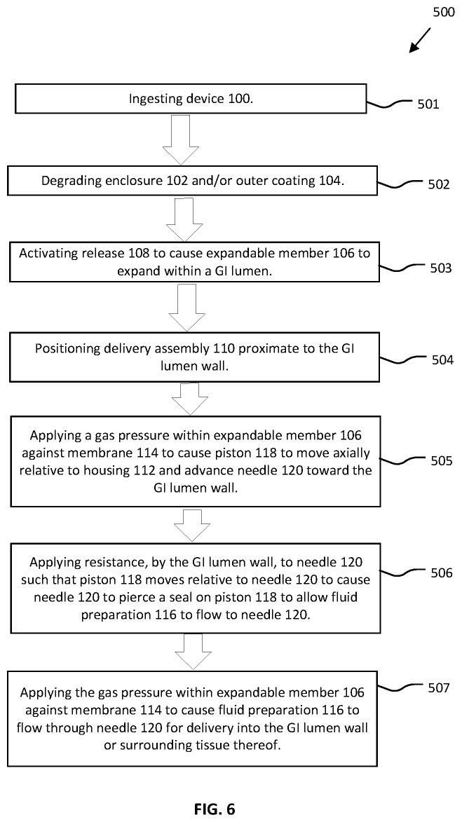

Release mechanism 306 (e.g., actuator, trigger) is coupled to (e.g., press-fit, snap-fit, adhered), or integrally formed with, housing 302 within piston chamber 302a′. As shown in FIG. 3, release mechanism 306 is coupled to an inner surface of housing perimeter wall 302a within piston chamber 302a′. As discussed below, release mechanism 306 is structured to interface with piston 319 to substantially prevent advancement of piston 319 and needle 320 until sufficient pressure is generated by the gas within interior 202a of balloon 202. For example, FIG. 5 illustrates a cross-sectional view of release mechanism 306 taken along line 5-5 in FIG. 3. As shown in FIG. 5, release mechanism 306 has a substantially circular cross-sectional shape and includes one or more fingers 306a extending radially inward toward longitudinal axis 302ab. Each of the fingers 306a extends radially inward in a cantilevered manner from a circumferential edge of release mechanism 306 to a free end located adjacent piston 319. As discussed below, fingers 306a are structured to interface with a protrusion 319c of piston 319 via an interference (e.g., overlapping) condition to substantially prevent needle 320 from piercing needle seal 304 until sufficient pressure is generated within interior 202a. When the internal gas pressure reaches a threshold value (e.g., upon full or partial inflation of balloon 202), protrusion 319c overcomes the interference condition with fingers 306a by causing fingers 306a to deflect away from piston 319 to thereby allow piston 319 to move axially along longitudinal axis 302ab toward needle seal 304, such that tissue piercing member 324 pierces needle seal 304.

According to other embodiments, the release mechanism may be defined by a detachable connection between piston 319 and housing 302. For example, delivery assembly 300 may include a rear cover coupled to housing 302. The rear cover may include one or more snap features which interface with a complementary feature on piston 319 to temporarily hold piston 319 relative to housing 302 until sufficient pressure is generated by the gas within interior 202a of balloon 202.

In another example, the rear cover may have a frangible (e.g., breakable) connection with piston 319, which may be structured to detach in response to a threshold gas pressure generated within interior 202a to permit axial movement of piston 319 relative to housing 302.

Piston 319 is movably (e.g., slidably) coupled to housing 302. In the unactuated state shown in FIG. 3, piston 319 is disposed in piston chamber 302a′. Piston 319 includes a piston perimeter wall 319a which defines a fluid cavity 319a′ and a needle cavity 319a″. As discussed below, membrane 315 is coupled to piston 319 such that membrane 315 encloses fluid cavity 319a′ to define reservoir 315a for containing fluid preparation 317 therein. Piston 319 is structured to move axially relative to housing 302 in response to a threshold gas pressure applied against an outer surface of membrane 315. Piston perimeter wall 319a further defines a fill port 319a″ for filling reservoir 315a with fluid preparation 317.

After filling (partially or fully) reservoir 315a, fill port 319a′″ is substantially fluidly sealed by, for example, heat staking housing perimeter wall 302a to substantially block fill port 319a′″. In other embodiments, a separate seal (e.g., silicone or aluminum foil) may be coupled to housing perimeter wall 302a at fill port 319a′″. In other embodiments, fill port 319a′″ may include a septum (e.g., silicone septum) such that fill port 319a′″ can self-seal after filling reservoir 315a with fluid preparation 317.

In any case, reservoir 315a may be filled with fluid preparation 317 before piston 319 is coupled to housing 302 and balloon 202. This can, advantageously, allow for flexibility relating to aseptic assembly of device 200. For example, prior to assembling piston 319 with housing 302 and balloon 202, a vacuum may be applied to reservoir 315a via fill port 319a′″ in an aseptic environment to substantially evacuate reservoir 315a. The evacuated reservoir 315a can then be filled with fluid preparation 317 via fill port 319a′″ in the aseptic environment prior to assembly with housing 302 and balloon 202.

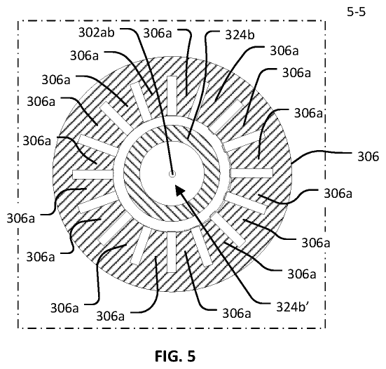

Still referring to FIG. 3, piston perimeter wall 319a further defines a stop feature 319b extending from an inner surface of piston perimeter wall 319a toward longitudinal axis 302ab within needle cavity 319a″. Stop feature 319b is structured to interface with a complementary feature on needle 320 (depicted as protrusion 326c in FIG. 3) to limit the axial travel of piston 319 relative to needle 320, the details of which are discussed below with reference to FIG. 4.

Piston perimeter wall 319a further defines protrusion 319c extending from an outer surface of piston perimeter wall 319a at an upper portion of needle cavity 319a″. In the unactuated state shown in FIG. 3, protrusion 319c is located adjacent release mechanism 306 below fingers 306a of release mechanism 306. As previously discussed with respect to FIG. 5, protrusion 319c interfaces with fingers 306a to substantially prevent advancement of needle 320 through needle seal 304 until the internal pressure generated by the gas within interior 202a reaches a threshold value. For example, the threshold pressure may be associated with a fully inflated state of balloon 202 within the GI lumen to ensure substantial alignment of elongated section 210 with the GI lumen wall before advancing needle 320 from housing 302.

Reservoir seal 307 is coupled (e.g., adhered) to an inner surface of piston perimeter wall 319a to define a substantially fluid-tight seal between fluid cavity 319a′ and needle cavity 319a″. In this way, reservoir seal 307 substantially prevents fluid preparation 317 from entering needle cavity 319a″ until needle 320 pierces reservoir seal 307. Reservoir seal 307 may be formed from a penetrable material such as aluminum foil.

Piston seal 323 is coupled to an outer portion of piston perimeter wall 319a by piston seal holder 321. Piston seal holder 321 is coupled (e.g., adhered, snap-fit) to an upper portion of piston perimeter wall 319a to hold piston seal 323 relative to piston 319. In other embodiments, piston seal 323 may be integrally formed with (e.g., insert molded), or otherwise coupled to, a peripheral side of piston 319. Piston seal 323 is structured to engage an inner surface of housing perimeter wall 302a to create a substantially fluid-tight seal between piston 319 and housing perimeter wall 302a within piston chamber 302a′. Piston seal 323 is further structured to allow for relative axial movement between piston 319 and housing 302. Piston 319 may be formed from a polymeric material (e.g., acrylonitrile butadiene styrene (ABS)), or other material or combinations of materials. Piston seal 323 may be formed from silicone or other suitable material.

Still referring to FIG. 3, membrane 315 is coupled to piston 302 to define reservoir 315a. For example, membrane 315 may be coupled to piston 319 by heat sealing, ultrasonic welding, adhering, or by other means. In the embodiment shown, membrane 315 is coupled to an inner surface of piston perimeter wall 319a which defines fluid cavity 319a′. Membrane 315 extends circumferentially about piston 319 to enclose fluid cavity 319a′. In this way, membrane 315, piston perimeter wall 319a, and reservoir seal 307 cooperatively contain fluid preparation 317 within reservoir 315a. Reservoir 315a may define a volume for containing up to about 250 μl or more of fluid, as discussed above with reference to FIG. 1. For example, reservoir 315a may define a volume for containing from about 50 μl to about 200 μl of fluid, including from 50 μl to 250 μl of fluid, such as 50 μl, 100 μl, 150 μl, or 200 μl of fluid, or any value therebetween, or may contain 200 μl or 250 μl of fluid, or any value therebetween. As discussed below, membrane 315 is structured to deform (e.g., bend, flex, constrict) in response to the gas pressure generated within balloon 202 (from reaction of first reactant 215 and second reactant 217) to expel fluid preparation 317 from reservoir 315a to needle 320. Membrane 315 may have a surface profile in a relaxed state (i.e., when reservoir 315a is unfilled with fluid preparation 317) that is complementary to an inner surface profile of piston 319 which defines fluid cavity 319a′. For example, membrane 315 may be vacuum formed using fluid cavity 319a′ as a mold for forming membrane 315 such that membrane 315 substantially mimics the inner surface profile of fluid cavity 319a′. In this way, membrane 315 can be sufficiently deformed to expel a substantial portion of fluid preparation 317 from reservoir 315a. Further, such a complementary shape of membrane 315 may help to facilitate sufficient evacuation of reservoir 315a for subsequent filling with fluid preparation 317 during assembly. Membrane 315 may be formed from a flexible polymeric material (e.g., polyethylene terephthalate (PET)), or other flexible material or combinations of materials having sufficiently low moisture and gas permeability properties for use with fluid preparation 317.

Needle 320 is movably (e.g., slidably) coupled to piston 319 and is disposed in needle cavity 319a″. In the embodiment shown in FIG. 3, needle 320 is collectively defined by needle sleeve 322, tissue piercing member 324, and seal piercing member 326. In other embodiments, needle 320 is a monolithic structure including needle sleeve 322, tissue piercing member 324, and seal piercing member 326.

Needle sleeve 322 couples tissue piercing member 324 to seal piercing member 326. Further, needle sleeve 322 functions to movably couple needle 320 to piston 319 within needle cavity 319a″. In the embodiment shown, needle sleeve 322 has a hollow cylindrical shape defining a needle sleeve first end 322a and an opposite needle sleeve second 322b. An outer surface of needle sleeve 322 engages with an inner surface of piston perimeter wall 319a within needle cavity 319a″ to define a substantially fluid-tight seal therebetween. In this manner, needle sleeve 322 can help to prevent fluid preparation 317 from passing through needle cavity 319a″ between needle 320 and piston perimeter wall 319a, such that a substantial amount of fluid preparation 317 can be directed from reservoir 315a through needle 320. Needle sleeve 322 is further structured to allow for relative axial movement between piston 319 and needle 320 along longitudinal axis 302ab. Needle sleeve 322 may be formed from, for example, silicone or other suitable material.

Tissue piercing member 324 is coupled (e.g., press-fit, adhered) to needle sleeve 322 at needle sleeve first end 322a. Tissue piercing member 324 extends outwardly from needle sleeve 322. Tissue piercing member 324 includes a tapered section 324a (e.g., pointed tip) which is structured to pierce though needle seal 304 and penetrate the GI lumen wall in response to sufficient axial movement of piston 319. Tissue piercing member 324 may have a length sufficient to penetrate through the GI lumen wall into the peritoneum or peritoneal cavity of a subject for delivery of fluid preparation 317 therein. The free end (e.g., pointed tip) of tapered section 324a defines a first end of needle 320. Tapered section 324a defines a needle opening 324a′ extending through tapered section 324a for discharging fluid preparation 317 into the GI lumen wall or surrounding tissue. Tissue piercing member 324 further includes an elongated section 324b extending from tapered section 324a. Elongated section 324b may be coupled to, or integrally formed with, tapered section 324a. A portion of elongated section 324b is disposed in needle sleeve 322 at needle sleeve first end 322a. Elongated section 324b defines a first needle channel 324b′ extending from needle opening 324a′ to an opposite end of elongated section 324b.

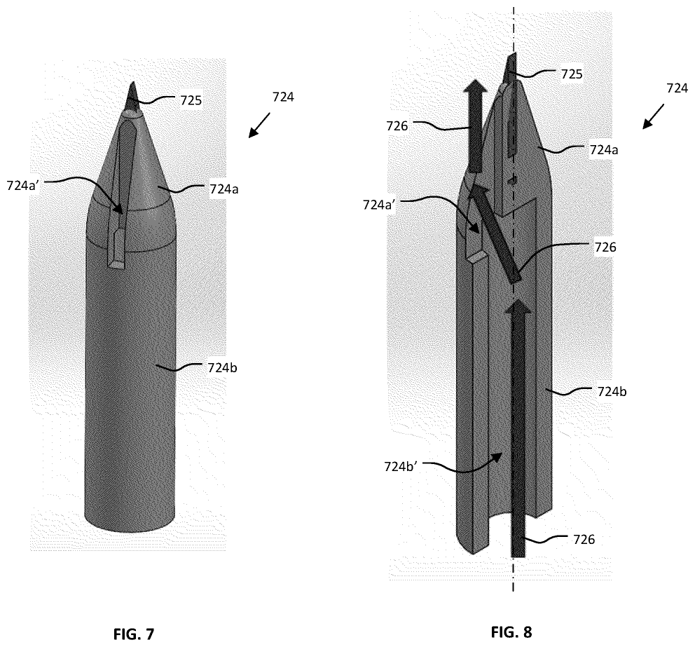

In other embodiments, tissue piercing member 324 includes a side opening (e.g., a slotted opening) for discharging fluid preparation 317 into the GI lumen wall or surrounding tissue. For example, referring to FIGS. 7-8, a tissue piercing member 724 (an embodiment of tissue piercing member 324) is shown according to another example. As shown in FIGS. 7-8, tissue piercing member 724 includes a tapered section 724a (e.g., frusto-conical) extending from elongated section 724b to a distal end or tip. Tapered section 724a includes a slotted opening 724a′ extending through a side wall of tapered section 724a to an interior channel 724b′ of elongated section 724b. Slotted opening 724a′ is defined by a recessed portion extending laterally through a side wall of tapered section 724a leading to a through hole extending to interior channel 724b′. Slotted opening 724a′ is positioned offset from the center axis and tip of tissue piercing member 724 to help avoid obstructing slotted opening 724a′ with bodily tissue or fluid which may accumulate therein upon tissue piercing member 724 penetrating a GI lumen wall or surrounding tissue.

The recessed portion of slotted opening 724a′ extending laterally through the side wall of tapered section 724a can, advantageously, help to direct the fluid preparation in a substantially longitudinal direction (represented by directional arrows 726 in FIG. 8) through tissue piercing member 724 to thereby avoid significantly changing the flow path through interior channel 724b′. In this manner, the fluid preparation can be efficiently discharged via tissue piercing member 724 into a GI lumen wall or surrounding tissue thereof.

Still referring to FIGS. 7-8, tissue piercing member 724 further includes a tip 725 coupled to (e.g., bonded), or integrally formed with (e.g., insert molded), tapered section 724a. Tip 725 defines an outermost distal end of tissue piercing member 724. Tip 725 is structured to pierce a GI lumen wall or surrounding tissue thereof. Tip 725 may be formed from a harder material than a material of tissue piercing member 724. For example, tip 725 may be formed from or include magnesium, metal, or another material or combinations of materials.

Tissue piercing members 324,724 are at least partially, or may be fully, degradable such that at least a portion of tissue piercing members 324,724 can substantially degrade within the GI lumen wall (or other location in the GI tract) upon delivery of fluid preparation 317. For example, tissue piercing members 324,724 may be formed from, or otherwise include, polyethylene oxide (PEO), magnesium, or other degradable material or combinations of materials.

Referring again to FIG. 3, seal piercing member 326 is coupled to (e.g., press-fit, adhered) needle sleeve 322. In the embodiment shown, seal piercing member 326 is structured as a substantially cylindrical member with a generally elongated configuration, although other shapes and configurations are contemplated according to other embodiments. Seal piercing member 326 extends from a seal piercing member first end 326a to an opposite seal piercing member second end 326b. Seal piercing member second end 326b defines a second end of needle 320 located opposite the first end. Seal piercing member first end 326a is coupled to (e.g., press-fit, adhered) needle sleeve 322 at needle sleeve second end 322b. Seal piercing member 326 extends outwardly from needle sleeve second end 322b and terminates at seal piercing member second end 326b. Seal piercing member second end 326b has a tapered profile (e.g., pointed end) which is structured to pierce reservoir seal 307 in response to relative movement between piston 319 and needle 320. Seal piercing member 326 further defines a protrusion 326c extending outwardly (e.g., radially) from an outer surface thereof. As discussed above and below, protrusion 326c functions as a complementary feature for interfacing with stop feature 319b to limit the axial travel of piston 319 relative to needle 320. Seal piercing member 326 further defines a second needle channel 326a′ for directing fluid preparation 317 from reservoir 315a to first needle channel 324b′ of tissue piercing member 324. Second needle channel 326a′ extends longitudinally along longitudinal axis 302ab from seal piercing member first end 326a to seal piercing member second end 326b. Seal piercing member 326 may be formed, for example, a polymeric material.

Referring to FIG. 4, a portion of device 200 including delivery assembly 300 is illustrated in an actuated state after device 200 has reached a desired location in the GI tract (e.g., the stomach or small intestine) for delivering fluid preparation 317. As shown in FIG. 6, balloon 202 has been inflated by a gas generated within interior 202a such that elongated section 210 is substantially aligned with a surface of the GI lumen wall. The generated gas within interior 202a applies a pressure (indicated by unidirectional arrows 330) against an outer surface of membrane 315 through a rear opening of housing 302. When the gas pressure reaches a threshold value (e.g., a pressure associated with a fully or partially inflated state of balloon 202), protrusion 319c overcomes the interference condition with release mechanism 306. As a result, piston 319 moves axially relative to housing 302 along longitudinal axis 302ab toward the GI lumen wall such that the first end of needle 320 (e.g., tapered section 324a) is advanced through needle seal 304 to contact the GI lumen wall.

Upon sufficient resistance from the GI lumen wall acting on needle 320, the gas pressure applied to membrane 315 causes piston 319 to move axially relative to needle 320 toward the GI lumen wall along longitudinal axis 302ab. For example, device 200 may be structured such that piston 319 moves relative to needle 320 upon needle 320 initiating contact with the GI lumen wall to generate sufficient resistance with the lumen wall. In other embodiments, device 200 may be structured such that piston 319 moves relative to needle 320 upon needle 320 penetrating a particular depth into the GI lumen wall to generate sufficient resistance with the lumen wall. This may be achieved by, for example, selecting a material of piston 319 and/or of needle sleeve 322 to have a sufficient coefficient of friction to control relative movement between piston 319 and needle 320. In either case, fluid preparation 317 remains in reservoir 315a until the second end of needle 320 (e.g., seal piercing member second end 326b) pierces reservoir seal 307 while the gas pressure in balloon 202 is applied against membrane 315. In this manner, delivery assembly 300 can allow for sequential timing between penetrating the GI lumen wall and discharging fluid preparation 317 to substantially avoid discharging fluid preparation 317 into the lumen environment and ensure delivery of fluid preparation 317 into the GI lumen wall or surrounding tissue (e.g., the peritoneum or peritoneal cavity).

As a result of sufficient movement of piston 319 relative to needle 320, the second end of needle 320 pierces reservoir seal 307 to allow fluid preparation 317 to flow from reservoir 315a to second needle channel 326a′. Piston 319 is permitted to move axially relative to needle 320 until stop feature 319b engages protrusion 326c. Upon engagement between stop feature 319b and protrusion 326c, the gas pressure applied against membrane 315 causes piston 319 and needle 320 to move axially relative to housing 302 along longitudinal axis 302ab such that needle 320 further penetrates the GI lumen wall or surrounding tissue.

In addition, upon piercing reservoir seal 307, the gas pressure applied against membrane 315 causes membrane 315 to deform inward relative to piston 319 toward needle 320 to expel fluid preparation 317 from reservoir 315a to needle 320. As a result, fluid preparation 317 is directed through second needle channel 326a′, first needle channel 324b′, and needle opening 324a′ into the GI lumen wall or surrounding tissue. In this manner, one or more therapeutic agents contained in fluid preparation 317 can be delivered into the subject's blood stream for systemic delivery.

Upon completion of delivery of fluid preparation 317, one or more components of needle 320 (e.g., tissue piercing member 324) can subsequently degrade within the GI lumen wall, or other area within the GI tract, along with one or more additional components of device 200 (e.g., housing 302, membrane 315, piston 319). In embodiments where needle 320 is substantially non-degradable, device 200 is structured to retract tissue piercing member 324 from the GI lumen wall within housing 302 upon completing delivery of fluid preparation 317. For example, device 200 may include a return spring or other biasing member coupled to needle 320 for retracting needle 320 within housing 302 after delivery of fluid preparation 317. In either of these embodiments, deflation valve 212 can release a substantial amount of the gas contained within interior 202a to allow for substantial deflation of balloon 202 and subsequent traversal of device 200 through the remainder of the GI tract to exit the anus of the subject.

Referring now to FIG. 6, a method 500 of delivering a fluid preparation 116 into the GI tract of a subject using device 100 is illustrated. In a first step, the subject ingests device 100 (e.g., by swallowing device 100) (Step 501.) As a result of ingestion, enclosure 102 and/or outer coating 104 are at least partially (or may be fully) degraded upon device 100 reaching a desired location in the GI tract for delivery of fluid preparation 116 (Step 502.) In response to degradation of enclosure 102 and/or outer coating 104, release 108 is activated to cause expandable member 106 to expand within the GI lumen (Step 503.) Expansion of expandable member 106 within the GI lumen causes delivery assembly 110 to be positioned proximate to the GI lumen wall (Step 504.) When a gas pressure within expandable member 106 reaches a threshold value, the gas pressure is applied against membrane 114 to cause piston 118 to move relative to housing 112 to advance needle 120 toward the GI lumen wall (Step 505.) The GI lumen wall applies a resistance to needle 120 such that piston 118 is moved axially relative to needle 120 to cause needle 120 to pierce a seal on piston 118 to allow fluid preparation 116 to flow to needle 120 (Step 506.) The gas pressure within expandable member 106 is applied against membrane 114 to expel fluid preparation 116 through needle 120 for delivery into the GI lumen wall or surrounding tissue thereof (Step 507.) In this manner, device 100 can deliver one or more therapeutic agents contained in fluid preparation 116 into the subject's blood stream for systemic delivery.

The foregoing description of various embodiments has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric and neonatal applications as well as various veterinary applications. Also, those skilled in the art will recognize, or be able to ascertain using no more than routine experimentation, numerous equivalents to the specific devices and methods described herein. Such equivalents are considered to be within the scope of the present disclosure.

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure as defined by the appended claims. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

1. A delivery assembly for an ingestible device for delivering a fluid preparation into a GI lumen wall or surrounding tissue thereof of a subject, the delivery assembly comprising:
a housing defining a chamber;
a piston movably disposed in the chamber;
a needle coupled to the piston; and
a membrane coupled to the piston such that the membrane and the piston cooperatively define a reservoir for containing the fluid preparation.

2. The delivery assembly of claim 1, wherein the piston defines a needle cavity, and wherein the needle is slidably coupled to the piston in the needle cavity.

3. The delivery assembly of claim 2, further comprising a seal coupled to the piston between the needle cavity and the reservoir to define a substantially fluid-tight seal therebetween.

4. The delivery assembly of claim 3, wherein the needle includes a first end structured to penetrate the GI lumen wall and a second end structured to pierce the seal in response to a resistance applied to the needle from the GI lumen wall.

5. The delivery assembly of claim 1, wherein the needle is structured to penetrate through the GI lumen wall into a peritoneum or peritoneal cavity of the subject for discharging the fluid preparation therein.

6. The delivery assembly of claim 1, wherein the membrane has a surface profile that is complementary to an inner surface profile of the piston which defines the reservoir.

7. The delivery assembly of claim 1, wherein the reservoir defines a volume for containing up to about 250 μl of fluid.

8. The delivery assembly of claim 1, wherein the reservoir defines a volume for containing about 50 μl to about 200 μl of fluid.

9. The delivery assembly of claim 1, wherein the housing is structured to be coupled to, and disposed in, an expandable member.

10. The delivery assembly of claim 1, wherein the piston is structured to move relative to the housing in response to a pressure applied against the membrane to advance the needle into the GI lumen wall.

11. The delivery assembly of claim 1, wherein the membrane comprises a flexible material to allow for deformation of the membrane by a pressure applied against an outer surface of the membrane to expel the fluid preparation from the reservoir into the needle.

12. The delivery assembly of claim 1, wherein the piston includes a fill port for filling the reservoir with the fluid preparation.

13. The delivery assembly of claim 1, further comprising a fluid preparation disposed in the reservoir, wherein the fluid preparation comprises at least one therapeutic agent.

14. The delivery assembly of claim 13, wherein the at least one therapeutic agent is one or more selected from an immunosuppressive drug, a chemotherapy drug, a central nervous system (CNS) drug, an antidiabetic drug, an enzyme replacement therapy (ERT) drug, an antibody, a hormone, insulin, an incretin or a combination thereof, or an oligonucleotide.

15-40. (canceled)

41. A delivery assembly for an ingestible device for delivering a fluid preparation into a GI lumen wall or surrounding tissue thereof of a subject, the delivery assembly comprising:
a housing defining a chamber;
a piston movably disposed in the chamber, the piston comprising a needle cavity and a fluid cavity;
a needle coupled to the piston at the needle cavity;
a membrane coupled to the piston at the fluid cavity such that the membrane and the piston cooperatively define a reservoir in the fluid cavity for containing the fluid preparation; and
a fluid preparation disposed in the reservoir, the fluid preparation comprising at least one therapeutic agent.

42. The delivery assembly of claim 41, wherein the needle is structured to penetrate through the GI lumen wall into a peritoneum or peritoneal cavity of the subject for discharging the fluid preparation therein.

43. The delivery assembly of claim 41, wherein the reservoir defines a volume for containing about 50 μl to about 200 μl of fluid.

44. The delivery assembly of claim 41, wherein the piston is structured to move relative to the housing in response to a pressure applied against the membrane to advance the needle into the GI lumen wall.

45. The delivery assembly of claim 41, wherein the membrane comprises a flexible material to allow for deformation of the membrane by a pressure applied against an outer surface of the membrane to expel the fluid preparation from the reservoir into the needle.

46. The delivery assembly of claim 41, wherein the at least one therapeutic agent is one or more selected from an immunosuppressive drug, a chemotherapy drug, a central nervous system (CNS) drug, an antidiabetic drug, an enzyme replacement therapy (ERT) drug, an antibody, a hormone, insulin, an incretin or a combination thereof, or an oligonucleotide.
